(19) United States
(12) Patent Application Publication
(10) Pub. No.: US 2019/0076127 A1
(43) Pub. Date: Mar. 14, 2019

(54) METHOD AND SYSTEM FOR AUTOMATICALLY SELECTING ULTRASOUND IMAGE LOOPS FROM A CONTINUOUSLY CAPTURED STRESS ECHOCARDIOGRAM BASED ON ASSIGNED IMAGE VIEW TYPES AND IMAGE CHARACTERISTIC METRICS

(71) Applicant: General Electric Company, Schenectady, NY (US)

(72) Inventors: Svein Arne Aase, Horten (NO); Kjetil Viggen, Trondheim (NO); Andreas Ziegler, Oslo (NO)

(21) Appl. No.: 15/701,683

(22) Filed: Sep. 12, 2017

(51) Int. Cl. A61B 8/08; A61B 5/0452; G06T 7/00; A61B 6/00

(52) U.S. Cl. CPC A61B 8/5215; A61B 5/0452; G06T 7/0012; G06F 19/321; A61B 8/48; A61B 8/0883; A61B 6/461

(57) ABSTRACT

A system and method for automatically selecting ultrasound image loops from a continuously captured stress echocardiogram is provided. The method may include continuously capturing ultrasound image data of a heart. The method may include separating the continuously captured ultrasound image data into image loops. Each of the image loops may have a predetermined number of heart cycles. The method may include automatically assigning an image view type from to at least a portion of the image loops. The method may include automatically assigning an image characteristic metric to each of the image loops having the assigned image view type. The method may include automatically presenting, at a display system, an image loop for each of the image view types based on the image characteristic metric.

### FIELD

Certain embodiments relate to ultrasound imaging. More specifically, certain embodiments relate to a method and system for automatically selecting ultrasound image loops from a continuously captured stress echocardiogram based on automatically assigned image view types and image characteristic metrics.

### BACKGROUND

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image. Stress echocardiograms are a type of ultrasound examination used to detect the presence of coronary artery disease. During a stress echocardiogram examination, ultrasound images of the heart are acquired before and after the heart is stressed by exercise or the injection of a drug. More specifically, ultrasound images of the heart are first obtained while a patient is at rest. Next, a patient may be asked to perform a physical activity, such as running on a treadmill or riding an exercise bicycle, to elevate the heart rate of the patient. Alternatively, a patient may be administered a drug, such as Dobutamine, to simulate exercise by elevating the patient's heart rate. The heart rate, blood pressure, and electrical activity of the heart may be monitored throughout the examination. Additional ultrasound images are acquired once a state of maximum stress on the heart is detected. The ultrasound images acquired at the resting state may be compared to ultrasound images acquired at the stressed state to assess the blood flow to the heart, which may be helpful is diagnosing coronary artery disease.

Typically, the window for acquiring ultrasound images after the heart of the patient reaches a state of maximum stress is one to two minutes in duration. Moreover, several image views of the patient's heart may be desired. Some ultrasound operators may continuously acquire ultrasound images at each of the desired image view positions over the one to two minute period that the heart of the patient is in the stressed state. Although the continuous capture of ultrasound image data may allow an ultrasound operator to easily acquire image data at each of the desired image view positions in the limited period of time, the amount of ultrasound image data acquired may be voluminous. Accordingly, an ultrasound operator may need to spend a considerable amount of time viewing well over one hundred image loops to select the most desirable image loop or loops at each image view type.

FIG. 1 is a screenshot of an exemplary conventional continuous capture stress echocardiogram image display 10, as known in the art. The image display 10 may be used in an exemplary conventional method for reviewing, selecting, and characterizing ultrasound image loops 20. Referring to FIG. 1, the continuous capture stress echocardiogram image display 10 may include several pages of image loops 20. For example, the continuous capture stress echocardiogram image display 10 of FIG. 1 shows page 11 of 31. The pages may be advanced or rewound using a page control mechanism 50. The pages may each display a number of image loops 20, such as the six image loops 20 shown in FIG. 1. The image loops 20 of FIG. 1 are identified as image loops 61 through 66 of a total 183 image loops 20. An ultrasound operator may use the page control mechanism 50 to scroll through the 31 pages of 183 total image loops 20. The ultrasound operator may manually assign view types with a view type selection user interface 30. For example, the view type selection user interface 30 may include available view types for manual selection by the ultrasound operator. The ultrasound operator may characterize the view type and/or select various images from the set of continuously captured image loops 20 for comparison with image loops acquired prior to the patient's heart being stressed and/or for any further suitable analysis. As shown with respect to FIG. 1, conventional methods for reviewing, selecting, and characterizing continuously captured ultrasound image loops 20 may be inefficient and time-consuming for an ultrasound operator. It may also be difficult and unreliable for an ultrasound operator to identify a best image for a particular view type because image loops 20 of different image view types may span multiple pages of the image display 10.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically selecting ultrasound image loops from a continuously captured stress echocardiogram, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a screenshot of an exemplary conventional continuous capture stress echocardiogram image display, as known in the art.

FIG. 2 is a block diagram of an exemplary ultrasound system that is operable to automatically select ultrasound image loops from a continuously captured stress echocardiogram, in accordance with various embodiments.

FIG. 3 is a screenshot of an exemplary continuous capture stress echocardiogram image display having automatically selected ultrasound image loops for a defined set of image view types, in accordance with exemplary embodiments.

FIG. 4 is a screenshot of an exemplary continuous capture stress echocardiogram image display including ultrasound image loops each having an automatically assigned image view type, in accordance with exemplary embodiments.

FIG. 5 is a flow chart illustrating exemplary steps that may be utilized for automatically selecting ultrasound image loops from a continuously captured stress echocardiogram, in accordance with various embodiments.

### DETAILED DESCRIPTION

Certain embodiments may be found in a method and system for automatically selecting ultrasound image loops from a continuously captured stress echocardiogram based on automatically assigned image view types and image characteristic metrics. Various embodiments have the technical effect of automatically assigning an image view type to at least a portion of continuously captured image loops using image recognition techniques and based on a predefined set of image view types. Moreover, certain embodiments have the technical effect of automatically assigning an image characteristic metric to ultrasound image loops based on associated image and heart acquisition data and/or image recognition techniques. Furthermore, various embodiments have the technical effect of automatically selecting and displaying, from continuously captured ultrasound image data, at least one image loop for each desired image view type based on a desired image characteristic metric.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as three-dimensional (3D) mode, B-mode, CF-mode, and/or sub-modes of B-mode and/or CF such as Shear Wave Elasticity Imaging (SWEI), TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

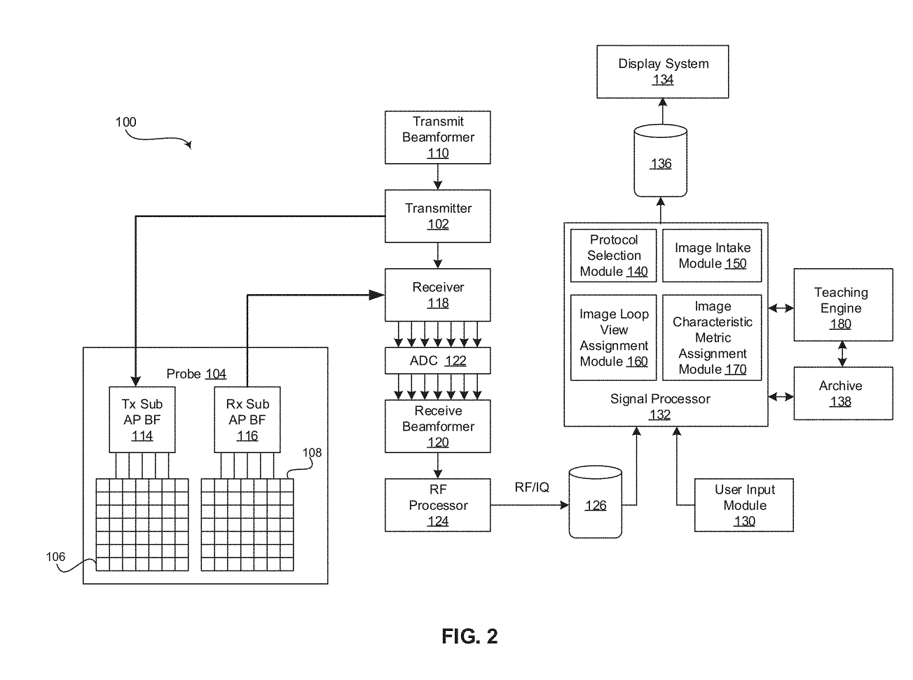

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 2.

FIG. 2 is a block diagram of an exemplary ultrasound system 100 that is operable to automatically select ultrasound image loops 220, 320 from a continuously captured stress echocardiogram, in accordance with various embodiments. Referring to FIG. 2, there is shown an ultrasound system 100. The ultrasound system 100 comprises a transmitter 102, an ultrasound probe 104, a transmit beamformer 110, a receiver 118, a receive beamformer 120, a RF processor 124, a RF/IQ buffer 126, a user input module 130, a signal processor 132, an image buffer 136, a display system 134, an archive 138, and a teaching engine 180.

The transmitter 102 may comprise suitable logic, circuitry, interfaces and/or code that may be operable to drive an ultrasound probe 104. The ultrasound probe 104 may comprise a three dimensional (3D) array of piezoelectric elements. The ultrasound probe 104 may comprise a group of transmit transducer elements 106 and a group of receive transducer elements 108, that normally constitute the same elements.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive and demodulate the signals from the receive sub-aperture beamformer 116. The demodulated analog signals may be communicated to one or more of the plurality of A/D converters 122.

The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the disclosure is not limited in this regard. Accordingly, in some embodiments, the plurality of A/D converters 122 may be integrated within the receiver 118.

The receive beamformer 120 may comprise suitable logic, circuitry, interfaces and/or code that may be operable to perform digital beamforming processing to, for example, sum the delayed channel signals received from the plurality of A/D converters 122 and output a beam summed signal. The resulting processed information may be converted back to corresponding RF signals. The corresponding output RF signals that are output from the receive beamformer 120 may be communicated to the RF processor 124. In accordance with some embodiments, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment, the RF processor 124 may comprise a complex demodulator (not shown) that is operable to demodulate the RF signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The user input module 130 may be utilized to input patient data, scan parameters, settings, select protocols and/or templates, approve automatically selected image loops, select alternative image loops, navigate image loop viewing, and the like. In an exemplary embodiment, the user input module 130 may be operable to configure, manage and/or control operation of one or more components and/or modules in the ultrasound system 100. In this regard, the user input module 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input module 130, the signal processor 132, the image buffer 136, the display system 134, the archive 138, and/or the teaching engine 180. The user input module 130 may include button(s), a touchscreen, motion tracking, voice recognition, a mousing device, keyboard, camera and/or any other device capable of receiving a user directive. In certain embodiments, one or more of the user input modules 130 may be integrated into other components, such as the display system 134, for example. As an example, user input module 130 may include a touchscreen display. In various embodiments, an ultrasound image presented at the display system 134 may be manipulated to zoom in and/or out in response to a directive received via the user input module 130.

The signal processor 132 may comprise suitable logic, circuitry, interfaces and/or code that may be operable to process ultrasound scan data (i.e., RF signal data or IQ data pairs) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform compounding, motion tracking, and/or speckle tracking. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. The processed image data can be presented at the display system 134 and/or may be stored at the archive 138. The archive 138 may be a local archive, a Picture Archiving and Communication System (PACS), or any suitable device for storing images and related information. In the exemplary embodiment, the signal processor 132 may comprise a protocol selection module 140, an image intake module 150, an image loop view assignment module 160, and an image characteristic metric assignment module 170.

The ultrasound system 100 may be operable to continuously acquire ultrasound scan data at a frame rate that is suitable for the imaging situation in question. Typical frame rates range from 20-70 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a protocol selection module 140 that comprises suitable logic, circuitry, interfaces and/or code that may be operable to configure the parameters associated with image acquisition and processing in response to user instructions provided at the user input module 130. For example, the protocol selection module 140 may receive a user input selecting a protocol and/or template defining a set of acquisition and/or processing parameters. As another example, the protocol selection module 140 may receive a user input selecting individual acquisition and/or processing parameters. In various embodiments, the selected protocol, template, and/or individual parameters may define the number of heart cycles per image loop 220, 320 and the desired image view types, among other things. For example, the number of heart cycles per image loop 220, 320 is typically one heart cycle per loop, but could be set at two cycles, three cycles, or any suitable number of cycles. Examples of image view types may include a parasternal long axis (PLAX) view, a parasternal short axis on mitral (SAX-MV) view, a four chamber (4CH) view, a two chamber (2CH) view, an apical long axis/3-chamber (APLAX) view, a parasternal short axis on papillary muscle (SAX-PM) view, or any suitable image view type. In a representative embodiment, the selected image acquisition and processing parameters may be stored for retrieval and application by the signal processor 132 and its associated modules 140-170 during ultrasound image acquisition and processing. For example, the parameters may be stored in archive 138 or any suitable data storage medium. In an exemplary embodiment, the protocol selection module 140 may apply a default protocol or template of default acquisition and processing parameters if the parameters are not defined or changed by user instructions provided via the user input module 130.

The signal processor 132 may include an image intake module 150 that comprises suitable logic, circuitry, interfaces and/or code that may be operable to create image loops 220, 320 from ultrasound image data continuously captured by the ultrasound system 100. For example, the image intake module 150 may apply the number of cycles per loop image processing parameter to determine the number of heart cycles to include in an image loop 220, 320. The image intake module 150 may analyze heart acquisition data associated with acquired ultrasound image data, such as the recorded heart rate, blood pressure, and electrical activity of the heart. The heart acquisition data associated with the acquired ultrasound image data may be referenced by the image intake module 150 to determine the ultrasound image data corresponding with each heart cycle that is chosen to generate each image loop 220, 320. In operation, the image intake module 150 separates the continuously captured stream of ultrasound image data into image loops 220, 320 each corresponding to the predefined number of heart cycles. In various embodiments, the image intake module 150 may apply image processing parameters to filter and/or otherwise enhance the visualization of the continuously acquired ultrasound image data.

The signal processor 132 may include an image loop view assignment module 160 that comprises suitable logic, circuitry, interfaces and/or code that may be operable to automatically assign an image view type to at least a portion of the image loops 220, 320 created by the image intake module 150. For example, the image loop view assignment module 160 may apply image detection mechanisms to automatically determine and characterize the image loops as one of the selected image view types defined by the protocol selection module 140. Additionally and/or alternatively, the image loop view assignment module 160 may characterize one or more image loops as unknown if the one or more image loops do not match one of the view type options. As an example, if an ultrasound operator is moving the probe 104 between image view types, or if the acquired ultrasound image data is obstructed by an operator finger or a rib of the patient, the ultrasound image data may not fit into one of the predefined image view types and/or may be unreadable.

In a representative embodiment, the image loop view assignment module 160 may include one or more deep neural networks and/or utilize any suitable form of machine learning processing functionality. For example, the image loop view assignment module 160 may be made up of an input layer, an output layer, and one or more hidden layers in between the input and output layers. Each of the layers may be made up of a plurality of processing nodes that may be referred to as neurons. For example, the input layer may have a neuron for each pixel or a group of pixels from an image loop 220, 320. The output layer may have a neuron corresponding to each image view type plus a neuron corresponding to an unknown or other image view type that does not match one of the predefined image view types. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to detected landmarks in the ultrasound image data. The processing performed by the image loop view assignment module 160 deep neural network may provide an image type assignment with a high degree of probability.

The signal processor 132 may include an image characteristic metric assignment module 170 that comprises suitable logic, circuitry, interfaces and/or code that may be operable to automatically assign an image characteristic metric to each image loop 220, 320, or to at least the image loops 220 320 having an assigned image view type 210, 310 matching one of the predefined image view types. The image characteristic metric assignment module 170 may assign the metric based on image quality characterizations and/or associated image or heart acquisition data, such as the acquisition time of the ultrasound image data forming the image loop 220, 320 and/or the heart rate information associated with the image loop, among other things. For example, the image characteristic metric assignment module 170 may provide the highest metric scores to the last acquired image loops 220, 320 for each image view type 210, 310 because an ultrasound operator typically moves on to the next image view type once the operator believes that the desired ultrasound image data was acquired. As another example, the image characteristic metric assignment module 170 may provide the highest metric scores to image loops 220, 320 associated with the highest and/or most stable heart rates because the image loops 220, 320 at these heart rates may be considered the best representations of the stressed state of the patient's heart. Furthermore, the image characteristic metric assignment module 170 may provide the highest metric scores to image loops 220, 320 having the highest image quality, such as images having the least noise, artifacts, shadows, smearing, haze, and the like. In various embodiments, the image characteristics metric assignment module 170 incorporates a plurality of acquisition time, heart rate, image quality, and/or any suitable factor to assign the metric to the image loops 220, 320. In an exemplary embodiment, the factors may be weighted if multiple factors are employed to determine the metric.

In a representative embodiment, the image characteristic metric assignment module 170 may include one or more deep neural networks and/or utilize any suitable form of deep learning processing functionality for scoring the quality of the image loops and/or combining weighted factors to provide the characteristic metric. For example, a deep neural network configured to score an image quality that may be used in determining an image characteristic metric may include a an input layer, an outer layer and one or more hidden intermediary layers. The input layer may include a neuron for each pixel or a group of pixels from an image loop 220, 320. The output layer may have a neuron corresponding to each available image quality score. Each neuron of each layer may perform a processing function and pass the processed ultrasound image information to one of a plurality of neurons of a downstream layer for further processing. As an example, neurons of a first layer may learn to recognize a blocked ultrasound transducer aperture, such as by an ultrasound operator finger, a rib of a patient, or any suitable obstruction. The neurons of a second layer may learn to recognize shadows in ultrasound image data caused by a patient's ribs or a breathing lung, among other things. The neurons of a third layer may learn to recognize movement noise caused by the probe 104 transitioning from one image view type to another image view type. The processing performed by the image characteristic metric assignment module 170 deep neural network(s) may provide an image quality score that may be used alone or with other factors in a weighted or non-weighted manner to determine the image characteristic metric that is assigned to the image loops 220, 320.

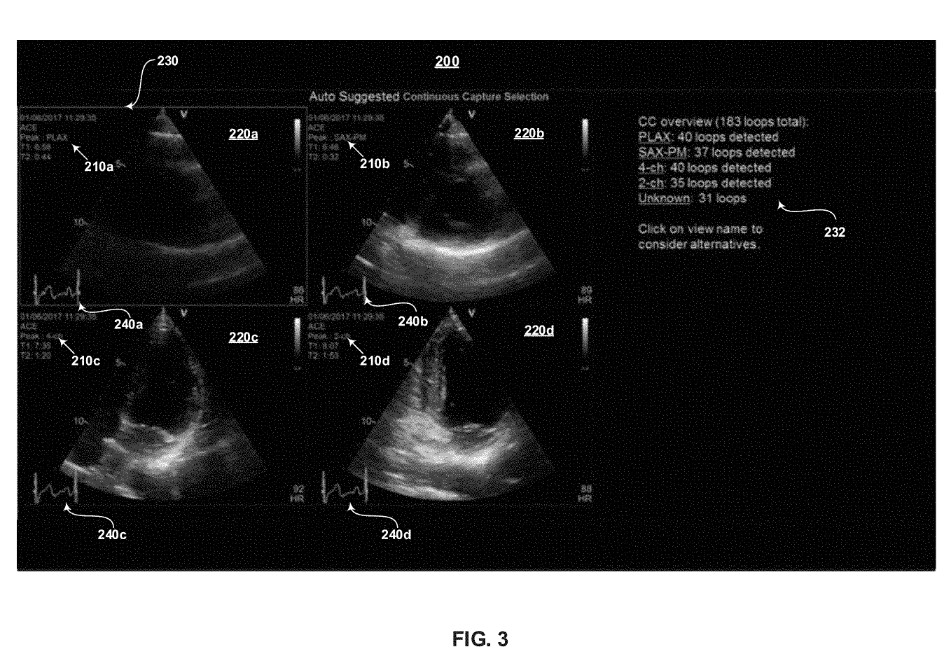

The image characteristic metric assignment module 170 includes suitable logic, circuitry, interfaces and/or code that may be operable to automatically select and display at least one image loop 220 of each image view type based on the assigned characteristic metric. For example, the image loop(s) 220 for each image view type having the most desirable metric may be automatically selected and presented at the display system 134 for operator approval based on the assignment of the image loop view 210, 310 by the image loop view assignment module 160 and the assignment of the image characteristic metric by the image characteristic metric assignment module 170. FIG. 3 is a screenshot of an exemplary continuous capture stress echocardiogram image display 200 having automatically selected ultrasound image loops 220a-d for a defined set of image view types 210a-d, in accordance with exemplary embodiments. Referring to FIG. 3, the continuous capture stress echocardiogram image display 200 comprises four automatically suggested ultrasound image loops 220a-d corresponding with four predefined image view types 210a-d. For example, the image display 200 shows a suggested image loop 220a corresponding with a parasternal long axis (PLAX) image view type 210a. The image display 200 shows a suggested image loop 220b corresponding with a parasternal short axis on papillary muscle (SAX-PM) image view type 210b. The image display 200 shows a suggested image loop 220c corresponding with a four chamber (4CH) image view type 210c. The image display 200 shows a suggested image loop 220d corresponding with a two chamber (2CH) image view type 210d. The image characteristic metric assignment module 170 may select and present the image loops 220a-d at the image display 200 based at least in part on the image view type assigned by the image loop view assignment module 160 and the metric assigned by the image characteristic metric assignment module 170. In various embodiments, each of the ultrasound image loops 220a-d may be linked to associated image or heart acquisition data 240a-d, such as an acquisition time, heart rate, blood pressure, electrical heart activity, and the like, all or some of which may be presented at the continuous capture stress echocardiogram image display 200.

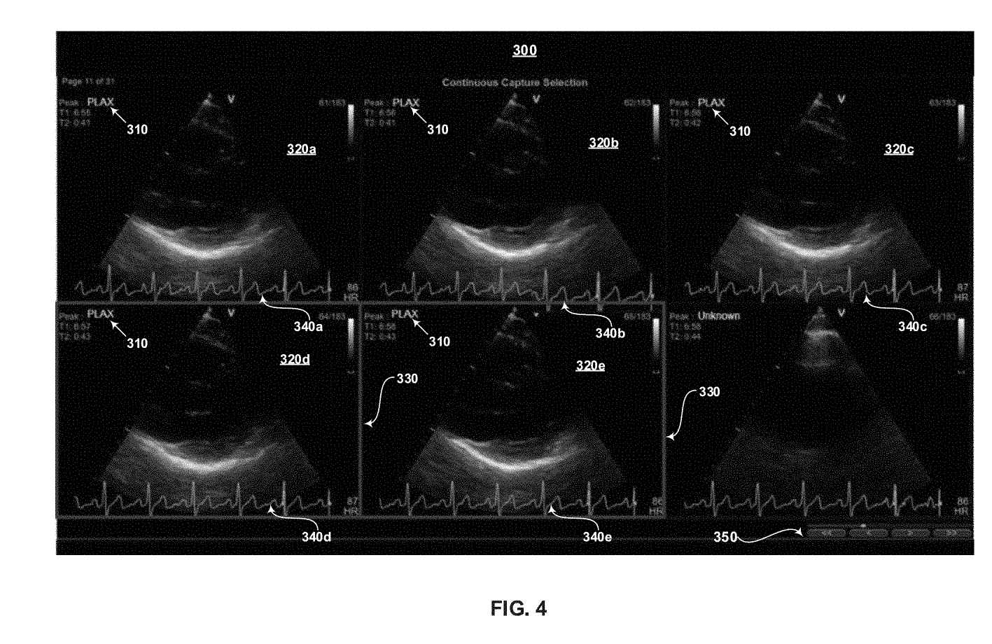

In an exemplary embodiment, an operator may interact with the image display 200 via the user input module 130 to approve the automatically selected image loops 220a-d and/or to access and view non-suggested image loops corresponding with any of the predefined image view types 210a-d and/or an unknown view type. For example, an operator may approve one or more of the automatically selected image loops 220a-d. In certain embodiments, any of the automatically selected image loops 220a-d approved by an operator may be stored at archive 138 and/or provided for further analysis, such as for comparison with resting heart rate ultrasound images of the echocardiogram examination. As another example, an operator may select one of the image view types 210a-d by clicking on or otherwise maneuvering a user input module 130 to choose one of the suggested image loops 220a-d. The selection may be visualized in the image display 200 by a box or other marker 230 highlighting an image loop 220a. The selection may navigate the image display 200 to an image display 300 having a plurality of image loops 320a-320e corresponding to the image view type 210a, 310 of the selected image loop 220a as discussed below with reference to FIG. 4.

Still referring to FIG. 3, the continuous capture stress echocardiogram image display 200 may comprise selectable image loop information 232 that may include textual information, or any suitable information, with selectable links, buttons, drop down menus, and/or the like for navigating to non-suggested image loops associated with a particular image view type. For example, the selectable image loop information 232 may provide information regarding the predefined and other image view types, such as the number of total image loops and the number of image loops for each view type. The selectable image loop information 232 shown in FIG. 3 identifies 183 total image loops including 40 detected PLAX loops, 37 detected SAX-PM loops, 40 detected 4CH loops, 35 detected 2CH loops, and 31 unknown/other loops. In various embodiments, the selectable image loops information 232 may be provided by the image loop view assignment module 160 that assigns at least a portion of the image loops 220, 320 with an image view type as discussed above. In a representative embodiment, the image view types identified in the selectable image loop information 232 may be selected to navigate the image display 200 to an image display 300 having a plurality of image loops 320a-320e corresponding to the selected image view type as discussed below with reference to FIG. 4. Additionally and/or alternatively, the image displays 200, 300 can be separately displayed at different image display systems 134 and/or screens. For example, selection 230, 232 of a particular image view type 210a-d in the overall selected image display 200 may launch the view specific image display 300 on a separate touch panel 130, 134 of the ultrasound system 100 while maintaining the overall selected image display 200 at the display system 134.

FIG. 4 is a screenshot of an exemplary continuous capture stress echocardiogram image display 300 including ultrasound image loops 320a-e each having an automatically assigned image view type 310, in accordance with exemplary embodiments. Referring to FIG. 4, the continuous capture stress echocardiogram image display 300 may be configured to present a plurality of image loops 320a-e corresponding with an image view type 310 selected from the image display 200 of FIG. 3. Still referring to FIG. 4, image loops 320a-e corresponding with a PLAX image view type 310 assigned by the image loop view assignment module 160 are presented at the image display 300. In various embodiments, the image loops 320a-e may be presented in a sorted order from the most desirable metric score to the least desirable metric score as assigned by the image characteristic metric assignment module 170. The continuous capture stress echocardiogram image display 300 may include a page control mechanism 350 operable to navigate through pages of the image loops in response to operator instruction received via a user input module 130. In an exemplary embodiment, each of the ultrasound image loops 320a-e may be linked to associated image or heart acquisition data 340a-e, such as an acquisition time, heart rate, blood pressure, electrical heart activity, and the like, all or some of which may be presented at the continuous capture stress echocardiogram image display 300. The image display 300 may include selection markers 330, such as boxes or any suitable marker for identifying user selected image loop(s) associated with the image view type 310. In various embodiments, a selection marker 330 may initially identify the automatically selected image loop 220a from the overall selected image display 200 of FIG. 3 in the view specific image display 300 of FIG. 4. An operator may, for example, confirm the automatic selection, deselect the automatically selected image loop, select a different image loop, and/or select additional image loops. The selection marker(s) 330 may identify the changes and/or confirmation of the selected image loop(s). In certain embodiments, the view specific image display 300 shown in FIG. 4 may be navigated by the operator to the overall selected image display 200 shown in FIG. 3 updated with the user-selected image loop(s) for approval of all automatically and/or manually selected image loops 220a-d for each image view type 210a-d. The approved image loops 220a-d may be stored at archive 138 and/or provided for further analysis, such as for comparison with resting heart rate ultrasound images of the echocardiogram examination.

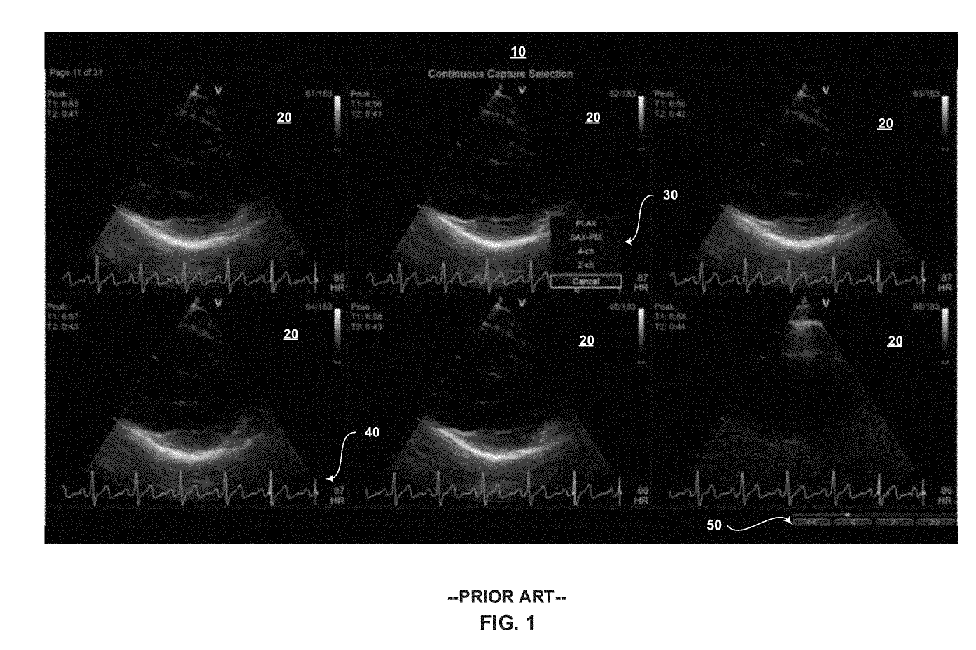

Referring again to FIG. 1, the teaching engine 180 may comprise suitable logic, circuitry, interfaces and/or code that may be operable to train the neurons of the deep neural network(s) of one or both of the image loop view assignment module 160 and the image characteristic metric assignment module 170. For example, the teaching engine 180 may train the deep neural networks of one or both of the image loop view assignment module 160 and image characteristic metric assignment module 170 using databases(s) of classified images. For example, an image loop view assignment module deep neural network may be trained by the teaching engine 180 with classified four chamber (4CH) images to identify characteristics and patterns in those images that are applied to identify the four chamber (4CH) image view type in non-classified images. As another example, an image characteristic metric assignment module deep neural network may be trained by the training engine 180 with images classified as having shadows caused by ribs of a patient to identify characteristics and patterns in those images that are applied to identify the occurrence of rib shadows in ultrasound images being scored for image quality. In various embodiments, the databases of training images may be stored in the archive 138 or any suitable data storage medium. In certain embodiments, the training engine 180 and/or training image databases may be external system(s) communicatively coupled via a wired or wireless connection to the ultrasound system 100.

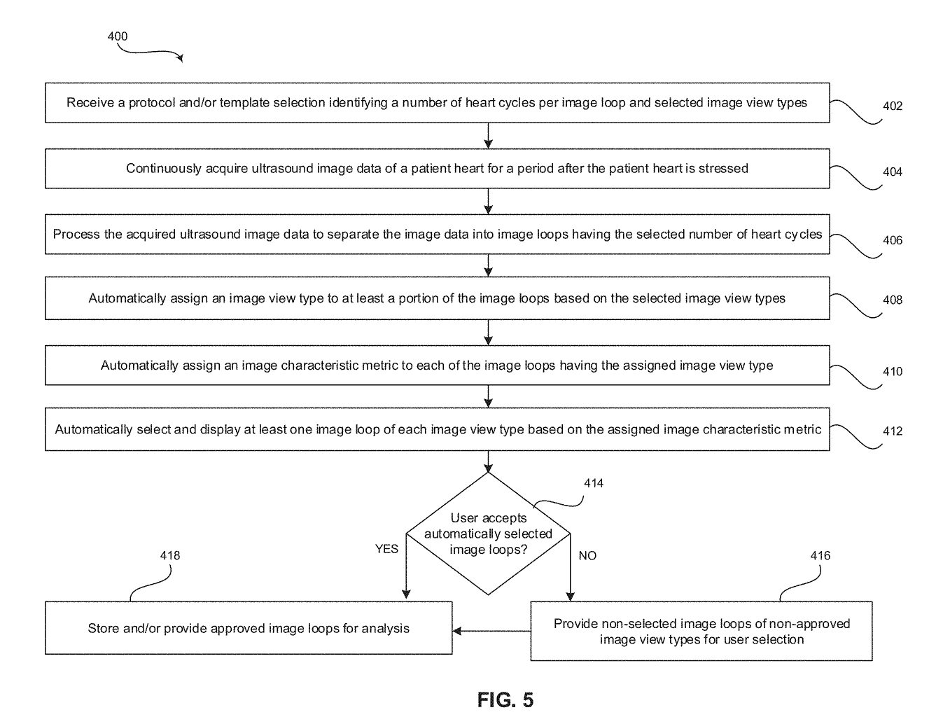

FIG. 5 is a flow chart illustrating exemplary steps 402-418 that may be utilized for automatically selecting ultrasound image loops 220 from a continuously captured stress echocardiogram, in accordance with various embodiments. Referring to FIG. 5, there is shown a flow chart 400 comprising exemplary steps 402 through 418. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 402, a signal processor 132 of an ultrasound system 100 may receive a protocol and/or template selection identifying a number of heart cycles per image loop and selected image view types. For example, a protocol selection module 140 of a signal processor 132 may receive operator instructions provided via a user input module 130 for defining and/or modifying image acquisition and processing parameters. The image acquisition and processing parameters may include a parameter defining the number of heart cycles per image loop. For example, an operator may define that each image loop includes one, two, three, or any suitable number of heart cycles. The image acquisition and processing parameters may include a parameter defining the image view types. For example, an operator may define that the echocardiogram examination includes PLAX, SAX-PM, 4CH, and 2CH image view types, or any suitable combination of image view types. In various embodiments, the protocol selection module 140 may apply default or most recently used protocols, templates, and/or parameters if the protocol selection module 140 does not receive user input defining and/or modifying the image acquisition and processing parameters.

At step 404, an ultrasound system 100 may continuously acquire ultrasound image data of a patient heart for a period after the patient heart is stressed. For example, stress echocardiogram examinations involve the acquisition of ultrasound images of the heart before and after a heart of a patient is stressed. The period for acquiring ultrasound images after the heart of the patient reaches a state of maximum stress by exercise or the injection of medicine is one to two minutes in duration. In various embodiments, the ultrasound system 100 may continuously capture image data at a number of different image view types as controlled by the operator of the ultrasound probe 104 during the one to two minute period that the heart of the patient is stressed.

At step 406, the signal processor 132 may process the acquired ultrasound image data to separate the image data into image loops 220, 320 having the selected number of heart cycles. For example, an image intake module 150 of the signal processor may generate image loops 220, 320 having the number of heart cycles defined by the protocol selection module 140 at step 402 from the ultrasound image data continuously captured at step 404. The continuously captured ultrasound image data may have associated image and heart acquisition data, such as image data acquisition time, a recorded heart rate, blood pressure, electrical activity of the heart, and/or the like, that may be analyzed by the image intake module 150 to generate the image loops 220, 320. For example, the image acquisition data associated with the acquired ultrasound image data may be referenced by the image intake module 150 to determine the ultrasound image data corresponding with each heart cycle that is chosen to generate each image loop 220, 320. The continuously captured stream of ultrasound image data is separated by the image intake module 150 into the image loops 220, 320 each corresponding to the predefined number of heart cycles.

At step 408, the signal processor 132 may automatically assign an image view type 210, 310 to at least a portion of the image loops 220, 320 based on the selected image view types. For example, an image loop view assignment module 160 of the signal processor 132 may analyze each of the image loops 220, 320 created at step 406 to assign one of the image view types defined at step 402 or an unknown image view type to each of the image loops 220, 320. The image loop view assignment module 160 may include a deep neural network, or any suitable processing algorithms, operable to analyze the image loops 220, 320 and assign an image view type based on detected heart structure and a viewpoint in the ultrasound image data of the image loops 220, 320. The automatically assigned image view type 210a-d, 310a-e may be stored in association with the corresponding image loop 220a-d, 320a-e in archive 138 or any suitable data storage medium.

At step 410, the signal processor 132 may automatically assign an image characteristic metric to each of the image loops 220, 320 having the assigned image view type 210, 310. For example, an image characteristic metric assignment module 170 of the signal processor 132 may analyze each of the image loops 220, 320 and/or the image or heart acquisition data of each image loop 220, 320 having an image view type 210, 310 assigned at step 408 to assign the characteristic metric to each of the image loops 220, 320. The characteristic metric may be based on image quality characterizations and/or associated image or heart acquisition data, such as the acquisition time of the ultrasound image data forming the image loop 220, 320 and/or the heart rate information associated with the image loop, among other things. For example, the image characteristic metric assignment module 170 may provide the highest metric scores to image loops having one or more of the last acquisition time, the highest and/or most stable heart rate, and/or the highest image quality. Although the metric scores may be configured so that higher scores are more desirable or lower scores are more desirable, as referred to herein, the term “highest” as it relates to the metric is defined as “most desirable,” irrespective of the metric scoring configuration. In various embodiments, the image characteristic metric assignment module 170 may include a deep neural network, or any suitable processing algorithms, operable to analyze one or both of the associated image acquisition data and the image loops 220, 320, and assign the characteristic metric to each of the image loops 220, 320. The automatically assigned image characteristic metric may be stored in association with the corresponding image loop 220a-d, 320a-e in archive 138 or any suitable data storage medium.

At step 412, the signal processor 132 may automatically select and display at least one image loop 220a-d of each image view type 210a-d based on the assigned image characteristic metric. For example, the image characteristic metric assignment module 170 of the signal processor 132 may analyze the image characteristic metric assigned to each image loop 220, 320. The image loop 220, 320 for each image view type 210a-d having the most desirable characteristic metric may be automatically selected by the image characteristic metric assignment module 170 based on the assigned image characteristic metric and presented in a continuous capture stress echocardiogram image display 200 at the display system 134.

At step 414, the signal processor 132 may receive a user input specifying whether one or more of the automatically selected image loops 220a-d is accepted. For example, the image characteristic metric assignment module 170 of the signal processor 132 may receive an instruction via the user input module 130 for approving one or more of the automatically selected image loops 220a-d. As another example, the image characteristic metric assignment module 170 of the signal processor 132 may receive an instruction via the user input module 130 for viewing non-selected image loops 320a-e corresponding to one or more image view types 310 so that the operator may confirm the automatically selected image loop or manually select an alternative image loop.

At step 416, the signal processor 132 may provide non-selected image loops 320a-e of non-approved image view types 310a-e for user selection if one or more of the automatically selected image loops 220a-d is not approved by the user. For example, in response to received instruction to view image loops 320a-e corresponding with a non-approved image view type 210a, 310, the image characteristic metric assignment module 170 of the signal processor 132 may present image loops 320a-e corresponding with the non-approved image type 210a, 310 in a continuous capture stress echocardiogram image display 300 at the display system 134. In various embodiments, the image loops 320a-e may be presented at the image display 300 in a sorted order from the most desirable metric score to the least desirable metric score as assigned by the image characteristic metric assignment module 170. The continuous capture stress echocardiogram image display 300 may be navigated by an operator to view and select one or more ultrasound image loops 320a-e for approval. The user selected image loop(s) may be identified by selection markers 330, such as boxes or any suitable marker for identifying user selected image loop(s) associated with the image view type 310. In certain embodiments, the view specific image display 300 may be navigated by the operator to the overall selected image display 200 updated with the user-selected image loop(s) for approval of all automatically and/or manually selected image loops 220a-d for each image view type 210a-d.

At step 418, the signal processor 132 may store and/or provide the approved image loops 220a-d for analysis. For example, the image characteristic metric assignment module 170 of the signal processor 132 may store the approved image loops 220a-d at archive 138. As another example, the image characteristic metric assignment module 170 of the signal processor 132 may provide the approved image loops 220a-d for further analysis, such as for comparison with resting heart rate ultrasound images of the echocardiogram examination.

Aspects of the present disclosure provide a method 400 and system 100 for automatically selecting ultrasound image loops 220, 320 from a continuously captured stress echocardiogram based on automatically assigned image view types 210, 310 and image characteristic metrics. In accordance with various embodiments, the method 400 may comprise continuously capturing 404, by an ultrasound probe 104, ultrasound image data of a heart. The method 400 may comprise separating 406, by a processor 132, 150, the continuously captured ultrasound image data into a plurality of image loops 220, 320. Each of the plurality of image loops 220, 320 may have a predetermined number of heart cycles. The method 400 may comprise automatically assigning 408, by the processor132, 160, an image view type 210, 310 from a plurality of image view types to at least a portion of the plurality of image loops 220, 320. The method 400 may comprise automatically assigning 410, by the processor 132, 170, an image characteristic metric to each of the plurality of image loops 220, 320 having the assigned image view type 210, 310. The method 400 may comprise automatically presenting 412, by the processor 132, 170, an image loop 220 from the plurality of image loops for each of the plurality of image view types 210 at a display system 134 based on the image characteristic metric.

In an exemplary embodiment, the method 400 may comprise acquiring heart data 240, 340 associated with the continuously captured ultrasound image data. The heart data 240, 340 may comprise one or more of a heart rate, a blood pressure, and electrical activity of the heart. In a representative embodiment, the method 400 may comprise receiving 402 a parameter selection defining a number of heart cycles per image loop 220, 320. The separating the continuously captured ultrasound image data into the plurality of image loops 220, 320 is based at least in part on the heart data 240, 340 and the parameter selection defining the number of heart cycles per image loop 220, 320. In certain embodiments, the method 400 may comprise receiving 414, by the processor, one of an indication of approval of one of the image loop 220 automatically presented at the display system 134, or an indication of non-approval of one of the image loop 220 automatically presented at the display system 134. The method 400 may comprise storing 418, by the processor 132, 170, the one of the image loop 220 automatically presented at the display system 134 if the indication of approval is received. The method 400 may comprise presenting 416, by the processor 132, 170, at least a portion of the plurality of image loops 320 for an image view type 210a, 310 from the plurality of image view types 210a-d corresponding with the one of the image loop 220a if the indication of non-approval is received.

In various embodiments, the method 400 comprises receiving 402 a parameter selection defining the plurality of image view types. In an exemplary embodiment, the image view type 210, 310 automatically assigned to each of the plurality of image loops 220, 320 is determined by one or both of one or more machine learning algorithms, and at least one deep neural network having an input layer, one or more hidden layers, and an output layer each comprising a plurality of neurons. Each of the plurality of neurons of the output layer may correspond with one of the plurality of image view types 210, 310 defined by the parameter selection. In a representative embodiment, the image characteristic metric automatically assigned to each of the plurality of image loops 220, 320 is based on one or more of an acquisition time of the continuously captured ultrasound image data forming the image loop 220, 320, the heart data 240, 340, and an image quality of the image loop 220, 320. In certain embodiments, the image characteristic metric is determined by one or both of one or more machine learning algorithms, and at least one deep neural network having an input layer, one or more hidden layers, and an output layer each comprising a plurality of neurons. Each of the plurality of neurons of the output layer may correspond with a different score of the image characteristic metric.

Various embodiments provide a system 100 for automatically selecting ultrasound image loops 220, 320 from a continuously captured stress echocardiogram based on automatically assigned image view types 210, 310 and image characteristic metrics. The system 100 may comprise an ultrasound probe 104 configured to continuously capture ultrasound image data of a heart. The system 100 may comprise a processor 132, 150 configured to separate the continuously captured ultrasound image data into a plurality of image loops 220, 320. Each of the plurality of image loops 220, 320 may have a predetermined number of heart cycles. The system 100 may comprise a processor 132, 160 configured to automatically assign an image view type 210, 310 from a plurality of image view types to at least a portion of the plurality of image loops 220, 320. The system 100 may comprise a processor 132, 170 configured to automatically assign an image characteristic metric to each of the plurality of image loops 220, 320 having the assigned image view type 210, 310. The system 100 may comprise a processor 132, 170 configured to automatically present an image loop 220 from the plurality of image loops for each of the plurality of image view types 210 at a display system 134 based on the image characteristic metric.

In a representative embodiment, the system 100 may comprise heart data acquisition equipment operable to acquire heart data 240, 340 associated with the continuously captured ultrasound image. The heart data 240, 340 may comprise one or more of a heart rate, a blood pressure, and electrical activity of the heart. In certain embodiments, the processor 132, 140 is configured to receive a parameter selection defining a number of heart cycles per image loop. The processor 132, 150 is configured to separate the continuously captured ultrasound image data into the plurality of image loops 220, 320 based at least in part on the heart data 240, 340 and the parameter selection defining the number of heart cycles per image loop.

In various embodiments, the image characteristic metric is automatically assigned by the processor 132, 170 to each of the plurality of image loops 220, 320 based on one or more of an acquisition time of the continuously captured ultrasound image data forming the image loop 220, 320, the heart data 240, 340, and an image quality of the image loop 220, 320. In an exemplary embodiment, the image characteristic metric is determined by one or both of the processor executing one or more machine learning algorithms, and at least one deep neural network of the processor 132, 170 having an input layer, one or more hidden layers, and an output layer each comprising a plurality of neurons. Each of the plurality of neurons of the output layer may correspond with a different score of the image characteristic metric. In a representative embodiment, the image view type 210, 310 is automatically assigned to each of the plurality of image loops 220, 320 determined by one or both of the processor executing one or more machine learning algorithms, and at least one deep neural network of the processor 132, 160 having an input layer, one or more hidden layers, and an output layer each comprising a plurality of neurons. Each of the plurality of neurons of the output layer may correspond with one of the plurality of image view types 210, 310.

Certain embodiments provide a non-transitory computer readable medium having stored thereon, a computer program having at least one code section. The at least one code section is executable by a machine for causing the machine to perform steps. The steps 400 may include receiving 404 continuously captured ultrasound image data of a heart. The steps 400 may include separating 406 the continuously captured ultrasound image data into a plurality of image loops 220, 320. Each of the plurality of image loops 220, 320 may have a predetermined number of heart cycles. The steps 400 may include automatically assigning 408 an image view type 210, 310 from a plurality of image view types to at least a portion of the plurality of image loops 220, 320. The steps 400 may include automatically assigning 410 an image characteristic metric to each of the plurality of image loops 220, 320 having the assigned image view type 210, 310. The steps 400 may include automatically presenting 412 an image loop 220 from the plurality of image loops for each of the plurality of image view types 210 at a display system 134 based on the image characteristic metric.

In an exemplary embodiment, the steps 400 may include acquiring heart data 240, 340 associated with the continuously captured ultrasound image. The heart data 240, 340 comprises one or more of a heart rate, a blood pressure, and electrical activity of the heart. In a representative embodiment, the steps 400 may include receiving 402 a parameter selection defining a number of heart cycles per image loop. The separating the continuously captured ultrasound image data into the image loops 220, 320 may be based at least in part on the heart data 240, 340 and the parameter selection defining the number of heart cycles per image loop 220, 320.

In certain embodiments, the image characteristic metric automatically assigned to each of the plurality of image loops 220, 320 is based on one or more of: an acquisition time of the continuously captured ultrasound image data forming the image loop 220, 320, the heart data 240, 340, and an image quality of the image loop 220, 320. In various embodiments, the image characteristic metric is determined by one or both of one or more machine learning algorithms, and at least one deep neural network having an input layer, one or more hidden layers, and an output layer each comprising a plurality of neurons. Each of the plurality of neurons of the output layer may correspond with a different score of the image characteristic metric. In an exemplary embodiment, the image view type 210, 310 automatically assigned to each of the plurality of image loops 220, 320 is determined by one or both of one or more machine learning algorithms, and at least one deep neural network having an input layer, one or more hidden layers, and an output layer each comprising a plurality of neurons. Each of the plurality of neurons of the output layer may correspond with one of the plurality of image view types 210, 310.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically selecting ultrasound image loops from a continuously captured stress echocardiogram based on automatically assigned image view types and image characteristic metrics.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
continuously capturing, by an ultrasound probe, ultrasound image data of a heart;
separating, by a processor, the continuously captured ultrasound image data into a plurality of image loops, each of the plurality of image loops having a predetermined number of heart cycles;
automatically assigning, by the processor, an image view type from a plurality of image view types to at least a portion of the plurality of image loops;
automatically assigning, by the processor, an image characteristic metric to each of the plurality of image loops having the assigned image view type; and
automatically presenting, by the processor, an image loop from the plurality of image loops for each of the plurality of image view types at a display system based on the image characteristic metric.

2. The method according to claim 1, comprising acquiring heart data associated with the continuously captured ultrasound image data, wherein the heart data comprises one or more of a heart rate, a blood pressure, and electrical activity of the heart.

3. The method according to claim 2, comprising receiving a parameter selection defining a number of heart cycles per image loop, wherein the separating the continuously captured ultrasound image data into the plurality of image loops is based at least in part on the heart data and the parameter selection defining the number of heart cycles per image loop.

4. The method according to claim 1, comprising:
receiving, by the processor, one of:
an indication of approval of one of the image loop automatically presented at the display system, or
an indication of non-approval of one of the image loop automatically presented at the display system;
storing, by the processor, the one of the image loop automatically presented at the display system if the indication of approval is received; and
presenting, by the processor, at least a portion of the plurality of image loops for an image view type from the plurality of image view types corresponding with the one of the image loop if the indication of non-approval is received.

5. The method according to claim 1, comprising receiving a parameter selection defining the plurality of image view types.

6. The method according to claim 5, wherein the image view type automatically assigned to each of the plurality of image loops is determined by one or both of:
one or more machine learning algorithms, and
at least one deep neural network having an input layer, one or more hidden layers, and an output layer each comprising a plurality of neurons, and wherein each of the plurality of neurons of the output layer corresponds with one of the plurality of image view types defined by the parameter selection.

7. The method according to claim 2, wherein the image characteristic metric automatically assigned to each of the plurality of image loops is based on one or more of:
an acquisition time of the continuously captured ultrasound image data forming the image loop,
the heart data, and
an image quality of the image loop.

8. The method according to claim 7, wherein the image characteristic metric is determined by one or both of:
one or more machine learning algorithms, and
at least one deep neural network having an input layer, one or more hidden layers, and an output layer each comprising a plurality of neurons, and wherein each of the plurality of neurons of the output layer corresponds with a different score of the image characteristic metric.

9. A system comprising:
an ultrasound probe configured to continuously capture ultrasound image data of a heart; and
a processor configured to:
separate the continuously captured ultrasound image data into a plurality of image loops, each of the plurality of image loops having a predetermined number of heart cycles;
automatically assign an image view type from a plurality of image view types to at least a portion of the plurality of image loops;
automatically assign an image characteristic metric to each of the plurality of image loops having the assigned image view type; and
automatically present an image loop from the plurality of image loops for each of the plurality of image view types at a display system based on the image characteristic metric.

10. The system according to claim 9, comprising heart data acquisition equipment operable to acquire heart data associated with the continuously captured ultrasound image, wherein the heart data comprises one or more of a heart rate, a blood pressure, and electrical activity of the heart.

11. The system according to claim 10, wherein the processor is configured to receive a parameter selection defining a number of heart cycles per image loop, and wherein the processor is configured to separate the continuously captured ultrasound image data into the plurality of image loops based at least in part on the heart data and the parameter selection defining the number of heart cycles per image loop.

12. The system according to claim 10, wherein the image characteristic metric is automatically assigned by the processor to each of the plurality of image loops based on one or more of:
an acquisition time of the continuously captured ultrasound image data forming the image loop,
the heart data, and
an image quality of the image loop

13. The system according to claim 12, wherein the image characteristic metric is determined by one or both of:
the processor executing one or more machine learning algorithms, and
at least one deep neural network of the processor having an input layer, one or more hidden layers, and an output layer each comprising a plurality of neurons, and wherein each of the plurality of neurons of the output layer corresponds with a different score of the image characteristic metric.

14. The system according to claim 9, wherein the image view type is automatically assigned to each of the plurality of image loops determined by one or both of:
the processor executing one or more machine learning algorithms, and
at least one deep neural network of the processor having an input layer, one or more hidden layers, and an output layer each comprising a plurality of neurons, and wherein each of the plurality of neurons of the output layer corresponds with one of the plurality of image view types.

15. A non-transitory computer readable medium having stored thereon, a computer program having at least one code section, the at least one code section being executable by a machine for causing the machine to perform steps comprising:
receiving continuously captured ultrasound image data of a heart;
separating the continuously captured ultrasound image data into a plurality of image loops, each of the plurality of image loops having a predetermined number of heart cycles;
automatically assigning an image view type from a plurality of image view types to at least a portion of the plurality of image loops;
automatically assigning an image characteristic metric to each of the plurality of image loops having the assigned image view type; and
automatically presenting an image loop from the plurality of image loops for each of the plurality of image view types at a display system based on the image characteristic metric.

16. The non-transitory computer readable medium according to claim 15, comprising acquiring heart data associated with the continuously captured ultrasound image, wherein the heart data comprises one or more of a heart rate, a blood pressure, and electrical activity of the heart.

17. The non-transitory computer readable medium according to claim 16, comprising receiving a parameter selection defining a number of heart cycles per image loop, wherein the separating the continuously captured ultrasound image data into the image loops is based at least in part on the heart data and the parameter selection defining the number of heart cycles per image loop.

18. The non-transitory computer readable medium according to claim 15, wherein the image characteristic metric automatically assigned to each of the plurality of image loops is based on one or more of:
an acquisition time of the continuously captured ultrasound image data forming the image loop,
the heart data, and
an image quality of the image loop.

19. The non-transitory computer readable medium according to claim 15, wherein the image characteristic metric is determined by one or both of:
one or more machine learning algorithms, and
at least one deep neural network having an input layer, one or more hidden layers, and an output layer each comprising a plurality of neurons, and wherein each of the plurality of neurons of the output layer corresponds with a different score of the image characteristic metric.

20. The non-transitory computer readable medium according to claim 15, wherein the image view type automatically assigned to each of the plurality of image loops is determined by one or both of:
one or more machine learning algorithms, and
at least one deep neural network having an input layer, one or more hidden layers, and an output layer each comprising a plurality of neurons, and wherein each of the plurality of neurons of the output layer corresponds with one of the plurality of image view types.
